(19) United States
(12) Patent Application Publication
(10) Pub. No.: US 2010/0286229 A1
(43) Pub. Date: Nov. 11, 2010

(54) Modulation of Androgen Receptor for Treatment of Prostate Cancer

(76) Inventors: Katerina Gurova, Highland, OH (US); Andrei Gudkov, Gates Mills, OH (US)

Correspondence Address: POLSINELLI SHUGHART PC, 700 W. 47TH STREET, SUITE 1000, KANSAS CITY, MO, 64112-1802, US

(21) Appl. No.: 11/992,874

(22) PCT Filed: Sep. 29, 2006
(86) PCT No.: PCT/US06/38440

§ 371 Date: Jul. 15, 2010

Related U.S. Application Data
(60) Provisional application No. 60/722,467, filed on Sep. 30, 2005.

(51) Int. Cl. C12Q 1/68; C12N 1/19; C12N 5/10; A61K 31/713; A61P 35/00

(52) U.S. Cl. 514/44 A; 435/6; 435/254.2; 435/348; 435/325; 435/366; 435/353; 435/354; 435/367

(57) ABSTRACT

The present invention provides methods for the reduction of endotoxins in a plasmid preparation using a carbohydrate non-ionic detergent with silica chromatography.

### FIELD OF THE INVENTION

The invention generally relates to the modulation of cell signaling. More specifically, the invention relates to modulation of the androgen receptor.

### BACKGROUND OF THE INVENTION

Androgens are critical for the development and growth of normal prostate. Androgens are also responsible for the development of prostate diseases, including benign prostatic hyperplasia (BPH) and prostate cancer (PCa). The androgen receptor (AR) binds androgen ligands and transduces the signal in prostate cells to regulate the physiological and pathological development of the prostate gland. Signal transduction is characterized by translocation of the ligand-AR receptor complex into the nucleus followed by binding to an androgen response element (ARE), which regulate expression of androgen responsive genes. Conditions that activate abnormal AR trans-activation through AR mutations, amplification of AR, or androgen-independent signaling pathways can lead to or be a result of the development of prostatic diseases or androgen-refractory PCa.

In terms of androgen dependence, all CaP cells are divided into two categories: androgen sensitive (AS), for which proliferation is decreased in the absence of androgens; and androgen insensitive (AI), for which proliferation continues in the absence of androgens. In the absence of androgens, AS cells exhibit a decrease in both AR transcriptional activity and proliferation, although the former observation is controversial. Some studies report that androgen ablation leads to the decrease in AR transcriptional activity in these cells while they continue to proliferate. In some AI cells, AR transcription has been shown to be androgen-independent. It is unclear whether this is due to tumor cell type, the level of androgen-dependency, or to experimental design of measuring two parameters, transactivation and cell growth.

AI CaP cells can generally be subdivided into two groups: (i) cell lines that completely lose expression of AR (e.g., PC3, DU145 cells) and (ii) cell lines that maintain expression of AR, and which are wild type or are mutant and presumably retain AR signaling-dependence. The former cell lines do not seem to adequately reflect the situation for human tumors. In addition to the loss of AR expression, PC3 and DU145 cells do not express many prostate markers, including PSA and PSMA, known to be expressed in the prostate carcinoma.

Androgen ablation therapy is typically used to reduce AR ligand production or to block AR-mediated signaling. Antiandrogens have been used for treatment of PCa. Antiandrogens compete with 5α-DHT for AR binding, thereby blocking AR-mediated signaling. Unfortunately, almost all patients who show initially favorable responses to the androgen blockade eventually become refractory to antiandrogens. The acquisition of androgen independence by CaP during tumor progression rarely involves the loss of the androgen receptor (AR) signaling. On the contrary, androgen independence is associated with a maintenance of the transactivation function of AR in the absence of androgen. This may be achieved either by (i) the acquisition of activating mutations in AR causing constitutive activation or making AR responsive to non-androgen ligands; (ii) an amplification of the AR gene; or (iii) alterations in other components of the AR pathway.

Current therapeutic approaches for prostate cancer target only one possible mechanism of AR activation: inhibition of ligand-receptor interaction by either suppression of androgen production or by competitive blocking of testosterone binding by the receptor. Testosterone-dependent support of AR activity is, however, relevant only to hormone-dependent CaP. Therefore, current therapies may be effective against hormone-dependent CaP, but lose their efficacy when cancer transforms into an androgen-refractory form. The acquired resistance of prostate tumors to anti-androgen treatment and the ineffectiveness of current treatments demonstrates that there is a need to identify treatments that are effective against ligand-independent prostate tumors.

### SUMMARY OF THE INVENTION

A target cell is provided comprising a constitutively active mutant AR. The target cell may be substantially androgen-independent. The AR may be lacking the ligand binding domain of wtAR. The wtAR may comprise the sequence of SEQ ID NO: 2 or a sequence at least 80% identical thereto. The target cell may also comprise a second AR.

The mutant AR may be encoded by residues 1116-3878 of SEQ ID NO: 1 or a sequence at least 80% identical thereto. The mutant AR may be lacking the C-terminal 248 to 295 residues of SEQ ID NO: 2. The mutant AR may also be lacking the C-terminal 261 residues of SEQ ID NO: 2. The mutant AR may comprise residues 1-659 of SEQ ID NO: 2 or a sequence at least 80% identical thereto.

The target cell may comprise a reporter construct, which may comprise an expression control sequence. The expression control sequence may comprise a promoter, which may be a minimal promoter. The expression control sequence may also comprise an ARE.

The target cell may comprise a siRNA comprising a sequence substantially complementary to a gene encoding the mutant AR. The target cell may also comprises a siRNA comprising a sequence substantially complementary to a gene encoding the second AR, which is optionally not substantially complementary to a gene encoding the mutant AR.

A method for screening an agent for modulating AR activity is also provided. An agent may be contacted with the target cell, which may be substantially androgen-independent. A difference in the level of reporter produced by the cells in the presence of the agent compared to a control indicates that the agent is a modulator of AR activity.

A method of treating prostate cancer is also provided. A patient in need of treatment may be administered a composition comprising a modulator of AR signaling. The modulator may not affect ligand binding of AR. The patient may suffer from androgen refractory prostate cancer. The patient may also suffer from androgen-sensitive prostate cancer.

### BRIEF DESCRIPTION OF THE DRAWINGS

FIG. 1 demonstrates the androgen dependence of different prostate cancer cell lines. Panel A: Activity of luciferase under AR-responsive promoter (ARE-Luc) in LNCaP, C4-2 and CWR22R cells in media with FBS or CSS with or w/o DHT. Panel B: Growth of LNCaP and C4-2 cells in media with FBS or CSS with or w/o DHT.

FIG. 2 shows the generation and testing of the ARE-Luc reporter. Panel A: Schematic structure of ARE-Luc. Panel B: ARE-Luc reporter activity is dependent on the amount of ARcDNA transfected into 293 or Hela cells. Panel C: ARE-Luc reporter activity in cells with endogenous AR, with the box showing the amount of AR protein in each of the tested cell lines (Western blotting).

FIG. 3 shows the generation and testing of the ARΔLBD construct. Panel A: ARΔLBD construct. Panel B: Expression of wild type and mutant AR in HeLa cells (Western blotting). Panel C: ARE-Luc reporter activity in HeLa cells in steroid-free medium, transfected with wild type and ARΔLBD (normalized by CMV-b-gal transfection).

FIG. 4 demonstrates that DHT regulates the level of wild type but not ARΔLBD. Panel A: Activity of integrated ARE-Luc reporter in HeLa cells transduced with wild type, but not mutant AR, is dependent on DHT concentration. Panel B: Protein level of wild type or mutant AR in HeLa cells in FBS or CSS media with or without DHT. Panel C: Inhibition of proteasomes by PS341 leads to accumulation of wild type AR protein. Panel D: Immunofluorescent staining of HeLa cells with anti-AR antibodies in the presence or absence of DHT (1 nM). Panel E: Level of AR protein in different cells in FBS or CSS media with or w/o DHT.

FIG. 5 demonstrates that inhibition of AR expression interferes with growth of both androgen-dependent and independent CaP cells. Panel A: Anti-AR shRNAs (AR1, AR2, AR3) inhibit ARE-Luc reporter activity in LNCaP and C4-2 cells in cotransfection experiments compared to control shRNAs against GFP or FasL. Panels B and C demonstrate that expression of shRNA AR1 inhibits growth of CaP cells expressing AR. Panel B: retroviral transduction followed by hygromycin selection. Panel C: lentiviral transduction, 96 hours. DU145 and H1299 cells, which do not express AR, were used as a control.

FIG. 6 demonstrates that shRNAs against translated and non-translated regions of AR mRNA can suppress expression of only exogenous and/or endogenous AR. The ARE-Luc reporter was cotransfected together with siRNAs into C4-2 cells or plus pcDNA3-hAR into H1299 cells.

FIG. 7 shows the generation and testing of ligand independent versions of CWR22R cells. Panel A: CWR22R cells were infected with shAR6 lentivirus and then 48 hours later with ARDLBD lentivirus. Selection was done on puromycin (marker of shRNA lentivirus). Panel B: CWR22Rcells with shAR and ARDLBD do not respond to DHT by ARE-Luc reporter activation.

FIG. 8 indicates that CaP cells do not tolerate overexpression of AR. Panel A: Failure of selection of colonies over-expressing wild type of mutant AR. Panel B: AR protein was not overexpressed in rare colonies survived selection (Western blotting). Panel C: Lentiviral transduction of wild type and mutant AR into HeLa cells (Western blotting). Panel D: Loss of increased AR activity with time in CWR22R cells transduced with lentiviral wild type or mutant AR (virus with GFP was used as a control. Panel E: C4-2 cells were transduced with lentiviral AR or GFP and when kept in the medium with or without DHT (CSS) for indicated periods of time. Westerm Blotting with anti-AR or anti-actin antibodies. Panel F: An experiment similar to Panel E was done with CWR22R cells (data for 1, 2, 6, 10 days in DHT containing medium). Panel G: HT1080 cells were transduced with lentiviral AR or GFP. Cell lysates were prepared at 48 hours after transduction then used for Western blotting with indicated antibodies.

FIG. 9 shows the generation and testing of ARE-Luc reporter. A. Reporter was generated by insertion of 3 repeated androgen-responsive elements from the rat probasin promoter with flanking regions. The minimal promoter of Hsp70 gene was used. A luciferase-expressing cassette was flanked with two insulator (ins) elements to diminish the effect of integration sites. B. The responses of the ARE-Luc reporter on different levels of AR.

FIG. 10 shows the growth and AR-dependent transcription of different CaP cell lines in the presence of DHT. A. Growth curves of CaP cell measured by methylene blue staining. 10⁴ cells were plated in the wells of 12 well plates. B. Cells from the experiment described in A were lysed on the 8^{th} day after start; luciferase activity was measured in cell lysates and normalized to the amount of protein. Data are presented as fold of changes in reporter activity.

FIG. 11 shows the effect of shRNA constructs on ARE-Luc reporter activity in LNCaP cells. Cells were cotransfected with pARE-Luc and anti-AR shRNA constructs, shAR1, shAR2 and shAR3, or the control shRNA constructs shGFP and shFasL. At 48 hours, reporter activity was read from cell lysates. shRNAs were designed using the sequences as described in Example 4.

FIG. 12 shows the effect of AR knockdown on growth of CaP cells. A. Different cells were transduced with retroviruses expressing shRNAs against AR or GFP following selection on hygromycin. Cells colonies were stained with methylene blue. B. Normalization and quantification of the experiment shown on panel A. C-E. LNCaP cells were transfected with siRNA oligos against AR. At different time points cells were lysed and lysates were used for Western blotting with anti-AR antibodies (C), caspase activity (D, DEVDAse fluorescence) and cells death (E, calcein assay).

FIG. 13 shows that cells do not tolerate overexpression of AR. A. Photographs of plates of cells (methylene blue staining). Cells were transfected with either AR cDNA or empty vector and then selected on hygromycin. (v—vector, AR—AR cDNA).

FIG. 14 shows that overexpression of AR suppresses cell growth. A. Cells were transduced with high-titer lentiviruses encoding AR or GFP. Cells were stained with methylene blue 8 days after transduction. B. Analysis of cell cycle distribution of cells transduced with GFP or AR lentiviruses three days after transduction.

FIG. 15 shows that AR protein levels in cell transduced with lentiviral AR cDNA eventually decrease after transduction. A. Cells were transduced with AR or GFP lentiviruses, and at the indicated time points lysates of cells were used for Western blotting with anti-AR or anti-GAPDH antibodies. B. This experiment is similar to that described in FIG. 14A. 24 h after transduced cells were maintained in CSS containing medium with or without DHT (0.5 nM).

FIG. 16 shows that AR-dependent transcription induced by transduction of lentiviral AR eventually decreases after transduction. This experiment is similar to that of FIG. 15. Lysates of CWR22R ARE-Luc cells were used for the reporter assay.

FIG. 17 shows the effect of AR on p21 protein levels. Increased expression (A) or activity (B) of AR induces p21 levels. A. CWR22R cells. B. LNCaP cells.

FIG. 18 shows an alignment of full-length AR proteins from the following organisms: Homo sapiens (SEQ ID NO: 14); Pan troglodytes (SEQ ID NO: 15), Macaca mulatta (SEQ ID NO: 16); Canis familiaris (SEQ ID NO: 17); Mus musculus (SEQ ID NO: 18); Rattus norvegicus (SEQ ID NO: 19); Sus scrofa (SEQ ID NO: 20); Gallus gallus (SEQ ID NO: 21); Bos taurus (SEQ ID NO: 22); Danio rerio (SEQ ID NO: 23). The hinge region is indicated in black shading. The unshaded sequence C-terminal to the hinge region is the ligand binding domain.

### DETAILED DESCRIPTION

A new therapeutic strategy for the treatment of androgen independent (as well as androgen-dependent) prostate cancer is provided by targeting AR or components of the AR pathway downstream of ligand-receptor interaction. The therapeutic strategy is based on the prostate being a uniquely hormone-dependent tissue. The prostate may completely depend on the function of the androgen receptor, a transcription factor with a predominant expression in the prostate and the testis. CaP may originate from prostate epithelial cells and may never lose dependence on the function of AR. AR signaling may be a survival factor for both androgen-dependent and independent CaP. This makes AR an extremely attractive target for the development of an anti-prostate cancer therapy not only for hormone-dependent but also for hormone-refractory CaP types. A treatment for CaP is provided by modulating AR function downstream of AR-ligand interaction regardless of the supporting mechanism.

A cell-based bioassay is provided for screening agents that modulate AR-mediated activity. Candidate agents are exposed to target cells capable of growing in culture but which have been engineered to express an AR variant which is constitutively active. The cell based bioassay may thus be used as an experimental model of androgen-insensitive prostate cancer. The cell-based assay may allow the identification of agents eliciting a desired effect on AR signaling. The agents may target AR itself or other components of the AR pathway, including known and unknown AR co-regulators. The agents identified by the bioassay may also be directed against intrinsic transactivation function of AR. An engineered cell for use in the bioassay is also provided.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

“Complement” or “complementary” as used herein to refer to a nucleic acid may mean Watson-Crick (e.g., A-t/u and C-G) or Hoogsteen base pairing between nucleotides or nucleotide analogs of nucleic acid molecules.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

“Operably linked” used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Selectable marker” used herein may mean any gene which confers a phenotype on a host cell in which it is expressed to facilitate the identification and/or selection of cells which are transfected or transformed with a genetic construct. Representative examples of selectable markers include the ampicillin-resistance gene (Amp'), tetracycline-resistance gene (Td), bacterial kanamycin-resistance gene (Kang, zeocin resistance gene, the AURI-C gene which confers resistance to the antibiotic aureobasidin A, phosphinothricin-resistance gene, neomycin phosphotransferase gene (nptII), hygromycin-resistance gene, beta-glucuronidase (GUS) gene, chloramphenicol acetyltransferase (CAT) gene, green fluorescent protein (GFP)-encoding gene and luciferase gene.

“Stringent hybridization conditions” used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Treat” or “treating” as used herein when referring to protection of a mammal from a condition, may mean preventing, suppressing, repressing, or eliminating the condition. Preventing the condition involves treating the mammal prior to onset of the condition. Suppressing the condition involves treating the mammal after induction of the condition but before its clinical appearance. Repressing the condition involves treating the mammal after clinical appearance of the condition such that the condition is reduced or maintained. Elimination the condition involves treating the mammal after clinical appearance of the condition such that the mammal no longer suffers the condition.

### 2. Target Cells

A target cell is provided. The target cell may be any cell that is capable of mediating AR-based signaling, either natively or by the addition of AR signaling components including, but not limited to, a nucleic acid encoding AR, an ARE, and a reporter. The target cell may be substantially androgen independent or completely androgen independent.

a. Cells

The target cell may be a eukaryote, such as a yeast, insect cell or animal cell. The animal cell may be a mammalian cell, such as a human, rat or mouse cell. The target cell may also be an immortalized cell. The target cell may also be derived from a tumor. Representative examples of target cells include, but are not limited to, LNCaP, C4-2, CWR33, CWR22R, W746L, T882A, Hela, 293 and H1299.

b. Mutant AR

The target cell may comprise a nucleic acid encoding a mutant AR. The mutant AR may be constitutively active. The mutant AR may comprise a modification that leads to a decrease in ligand binding. The modification may be an insertion, deletion or substitution in the ligand binding domain, with respect to a wild type AR (wtAR). The modification may further comprise an insertion, deletion or substitution in the hinge region. The modification may be a deletion of the ligand binding domain. The modification may also be a deletion of the ligand binding domain and the hinge region.

Representative examples of wtAR are the human AR (SEQ ID NO: 2), which is encoded by nucleotides 1116-3878 of SEQ ID NO: 1 (NM00044|NM00044.2|GI:21322251); the sequences of SEQ ID NOS: 9-23; and sequences at least 70%, 75%, 80%, 85%, 90%, 95%, 96%, 97%, 98% or 99% identical thereto.

The mutant AR may be lacking the ligand binding domain and a portion of the hinge regions, which may be residues 625-672 of SEQ ID NO: 2. The mutant AR may be lacking the C-terminal 248 to 295 residues of SEQ ID NO: 2. The mutant AR may also be lacking the C-terminal 261 residues of SEQ ID NO: 2. The mutant AR may also comprise residues 1-659 of SEQ ID NO: 2 or a sequence at least 80% identical thereto.

The mutant AR may also comprise the sequence of SEQ ID NO: 9 lacking a ligand binding domain, which may be residues 690-919. The mutant AR may also comprise the sequence of SEQ ID NO: 9 further lacking a hinge region, which may be residues 631-689. The mutant AR may also comprise residues 1-630 of SEQ ID NO: 9 or a sequence at least 77% identical thereto.

The mutant AR may also comprise the sequence of SEQ ID NO: 10 lacking a ligand binding domain, which may be residues 673-902. The mutant AR may also comprise the sequence of SEQ ID NO: 10 further lacking a hinge region, which may be residues 603-672.

The mutant AR may also comprise the sequence of SEQ ID NO: 11 lacking a ligand binding domain, which may be residues 650-899. The mutant AR may also comprise the sequence of SEQ ID NO: 11 further lacking a hinge region, which may be residues 600-649.

The mutant AR may also comprise the sequence of SEQ ID NO: 12 lacking a ligand binding domain, which may be residues 666-895. The mutant AR may also comprise the sequence of SEQ ID NO: 12 further lacking a hinge region, which may be residues 608-665.

The mutant AR may also comprise the sequence of SEQ ID NO: 13 lacking a ligand binding domain, which may be residues 683-912. The mutant AR may also comprise the sequence of SEQ ID NO: 13 further lacking a hinge region, which may be residues 625-682.

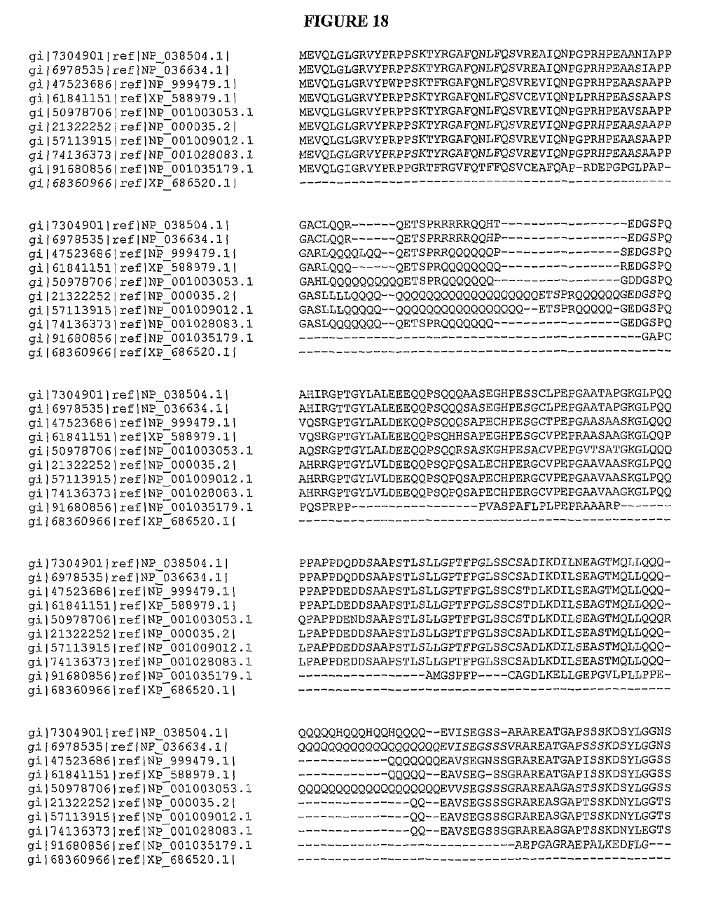

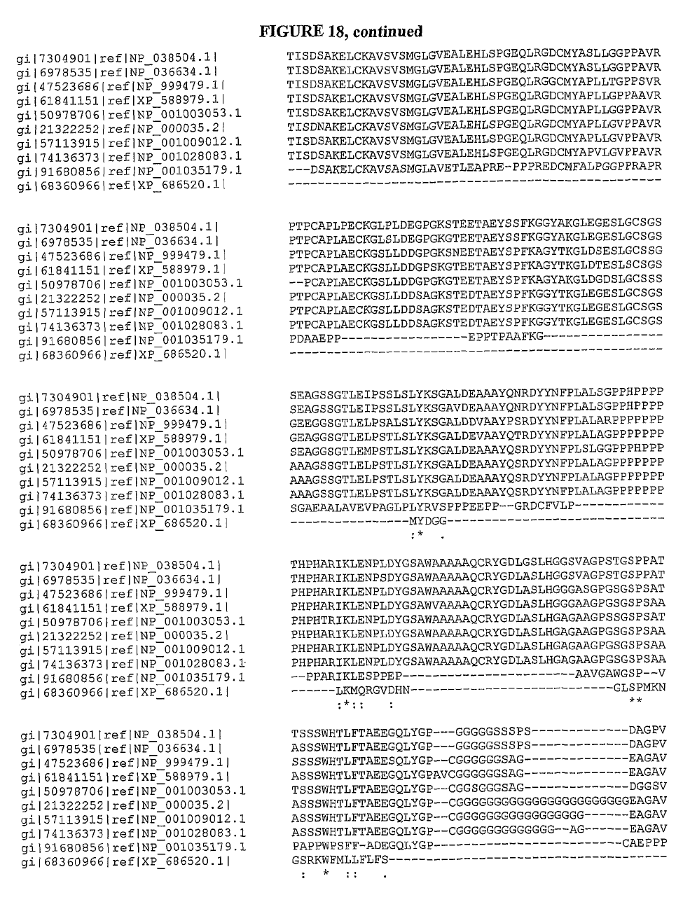

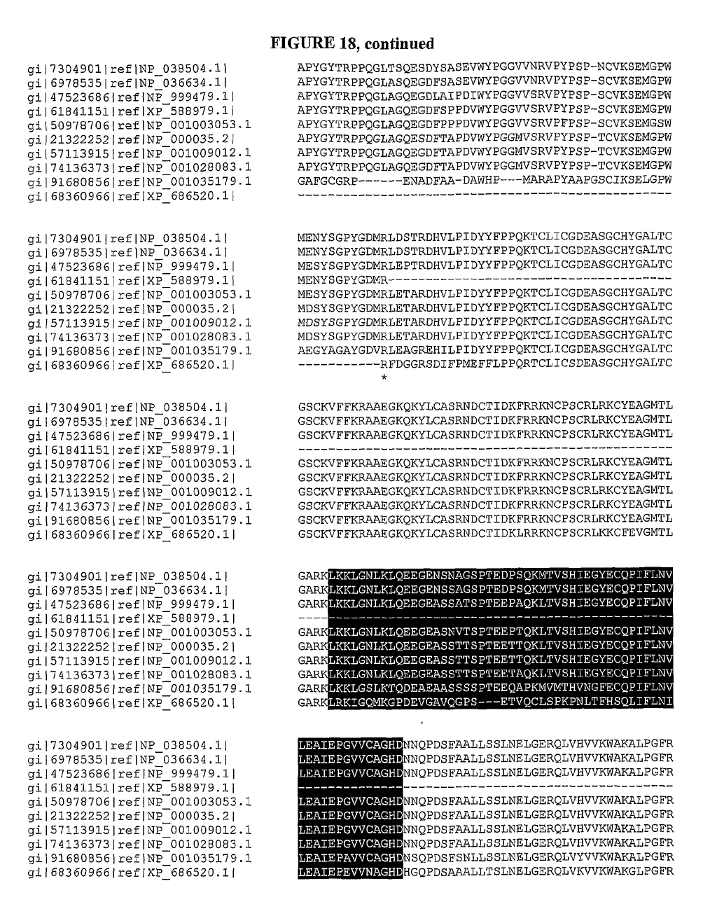

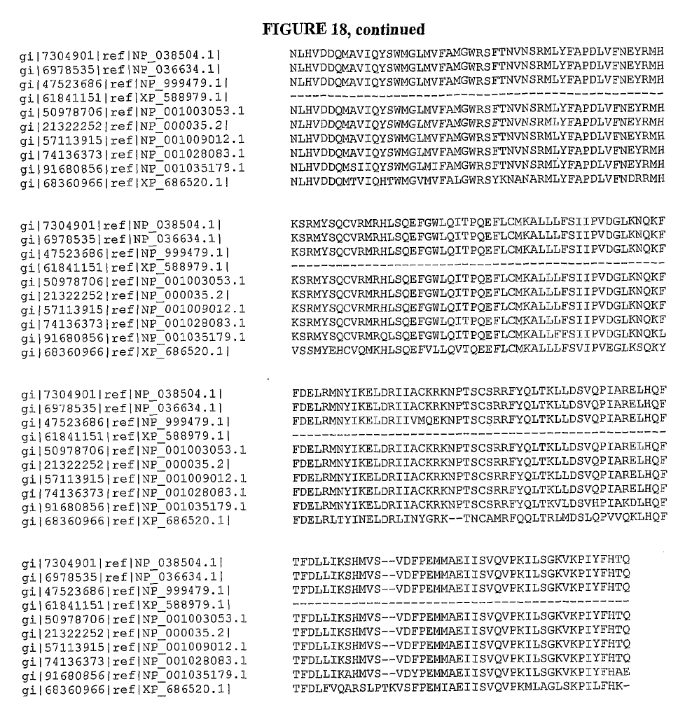

The mutant AR may also comprise a sequence selected from the group consisting of SEQ ID NOS: 14-23, lacking a ligand binding domain (indicated in FIG. 18 by unshaded residues C-terminal to the shaded region). The mutant AR may also comprise a sequence selected from the group consisting of SEQ ID NOS: 14-23, lacking both a ligand binding domain and a hinge region (indicated in FIG. 18 by unshaded residues C-terminal to the shaded region and the shaded region, respectively).

c. Second AR

The target cell may also comprise a second AR. The second AR may be a wtAR or mutant AR, a polypeptide at least 80%, 85%, 90%, 95%, 96%, 97%, 98% or 99% identical thereto.

d. Expression Construct

The target cell may also comprise an expression construct, which may allow monitoring of the status of AR-dependent transactivation. The expression construct may comprise an expression control sequence operatively linked to a nucleic acid and optionally an insulator. The nucleic acid may encode a reporter including, but not limited to, luciferase, GFP and CAT. The nucleic acid may also encode a polypeptide regulate by AR in vivo.

The expression control sequence may comprise a promoter, which may comprise a minimal promoter, such as Hsp70. The expression control sequence may also comprise an ARE, which may be derived from any AR regulated gene such as those described in Horie-Inoue et al., Biochem Biophys Res Commun, 24; 325(4):1312-7 (2004), which is incorporated herein by reference. The ARE may be derived from a promoter of a mammal, such as a human, mouse or rat. A representative example of an ARE may be found in the promoter from the rat probasin gene. Additional AREs include the following:

TABLE 1
AREs

| ARE | Sequence | SEQ ID NO |
|---|---|---|
| ARE-I | AGAACAGCAAGTGCT | 7 |
| ARE-II | GGATCAGGGAGTCTC | 8 |

The reporter construct may be maintained in a target cell as a plasmid. The reporter construct may also be introduced into the chromosome of a target cell. The reporter construct may also comprise a selectable marker, such as neo-resistance.

e. Regulation of AR Expression

Expression of AR in the target cell may be constitutive, inducible or repressible, which may be different for different forms of AR in the target cell. Expression of AR may be inducible using an inducible promoter known to those of skill in the art. Expression of AR may be repressible using RNAi silencing, such as antisense, siRNA, shRNA or miRNA.

The target cell may comprise a siRNA comprising a sequence that is substantially complementary to a gene encoding the mutant AR. The target cell may also comprise a siRNA comprising a sequence that is substantially complementary to a gene encoding the second AR and is optionally not substantially complementary to a gene encoding the mutant AR.

### 3. Assay

Methods of screening agents for modulating AR activity are also provided. An agent that modulates AR activity may be identified by contacting the agent with the target cell and determining the level of reporter produced by the cells in the presence of the agent compared to a control, which may be the level of the reporter without the agent. A modulator of AR activity may be identified by causing a decrease (inhibitor or AR activity) or increase (inducer of AR activity). The reporter may be an indicator gene, such as a gene encoded operatively linked to an expression control sequence comprising an ARE, or a phenotype associated with AR activity such as p53 activity, proliferation.

Candidate agents may be present within a library (i.e., a collection of compounds). Such agents may, for example, be encoded by DNA molecules within an expression library. Candidate agent be present in conditioned media or in cell extracts. Other such agents include compounds known in the art as “small molecules,” which have molecular weights less than 10⁵ daltons, preferably less than 10⁴ daltons and still more preferably less than 10³ daltons. Such candidate agents may be provided as members of a combinatorial library, which includes synthetic agents (e.g., peptides) prepared according to multiple predetermined chemical reactions. Those having ordinary skill in the art will appreciate that a diverse assortment of such libraries may be prepared according to established procedures, and members of a library of candidate agents can be simultaneously or sequentially screened as described herein.

The conditions under which a suspected modulator is added to a cell, such as by mixing, are conditions in which the cell may undergo apoptosis or signaling if essentially no other regulatory compounds are present that would interfere with apoptosis or signaling. Effective conditions include, but are not limited to, appropriate medium, temperature, pH and oxygen conditions that permit cell growth. An appropriate medium is typically a solid or liquid medium comprising growth factors and assimilable carbon, nitrogen and phosphate sources, as well as appropriate salts, minerals, metals and other nutrients, such as vitamins, and includes an effective medium in which the cell can be cultured such that the cell can exhibit apoptosis or signaling. For example, for a mammalian cell, the media may comprise Dulbecco's modified Eagle's medium containing 10% fetal calf serum.

Cells may be cultured in a variety of containers including, but not limited to tissue culture flasks, test tubes, microtiter dishes, and petri plates. Culturing is carried out at a temperature, pH and carbon dioxide content appropriate for the cell. Such culturing conditions are also within the skill in the art.

Methods for adding a suspected modulator to the cell include, but are not limited to, electroporation, microinjection, cellular expression (i.e., using an expression system including naked nucleic acid molecules, recombinant virus, retrovirus expression vectors and adenovirus expression), use of ion pairing agents and use of detergents for cell permeabilization.

### 4. Treatment

A method of treating prostate cancer is also provided. A patient in need of treatment may be administered a composition comprising a modulator of AR signaling. The modulator may not affect ligand binding of AR. The patient may suffer from androgen refractory prostate cancer. The patient may also suffer from androgen-sensitive prostate cancer.

a. Formulation

The compositions may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

The composition may also be liquid formulations including, but not limited to, aqueous or oily suspensions, solutions, emulsions, syrups, and elixirs. The composition may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

The compositions may also be formulated as suppositories, which may contain suppository bases including, but not limited to, cocoa butter or glycerides. Compositions of this invention may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. Compositions of this invention may also be formulated transdermal formulations comprising aqueous or nonaqueous vehicles including, but not limited to, creams, ointments, lotions, pastes, medicated plaster, patch, or membrane.

The compositions may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The compositions may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

b. Administration

The composition may be administered simultaneously or metronomically with other anti-cancer treatments such as chemotherapy and radiation therapy. The term “simultaneous” or “simultaneously” as used herein, means that the other anti-cancer treatment and the composition is administered within 48 hours, 24 hours, 12 hours, 6 hours, 3 hours or less, of each other. The term “metronomically” as used herein means the administration of the composition at times different from the chemotherapy and at certain frequency relative to repeat administration and/or the chemotherapy regiment.

The composition may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. The composition may also be administered in the form of an implant, which allows slow release of the composition as well as a slow controlled i.v. infusion.

c. Dosage

A therapeutically effective amount of an agent required for use in therapy varies with the nature of the condition being treated, the length of time that activity is desired, and the age and the condition of the patient, and is ultimately determined by the attendant physician. The desired dose may be conveniently administered in a single dose, or as multiple doses administered at appropriate intervals, for example as one, two, three, four or more subdoses per day. Multiple doses often are desired, or required.

When given in combination with other therapeutics, the composition may be given at relatively lower dosages. In addition, the use of targeting agents may allow the necessary dosage to be relatively low. Certain compositions may be administered at relatively high dosages due to factors including, but not limited to, low toxicity, high clearance, low rates of cleavage of the tertiary amine. As a result, the dosage of a composition may be from about 1 ng/kg to about 200 mg/kg, about 1 μg/kg to about 100 mg/kg, or about 1 mg/kg to about 50 mg/kg. The dosage of a composition may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

The present invention has multiple aspects, illustrated by the following non-limiting examples.

### Example 1

### AR Reporter Construct

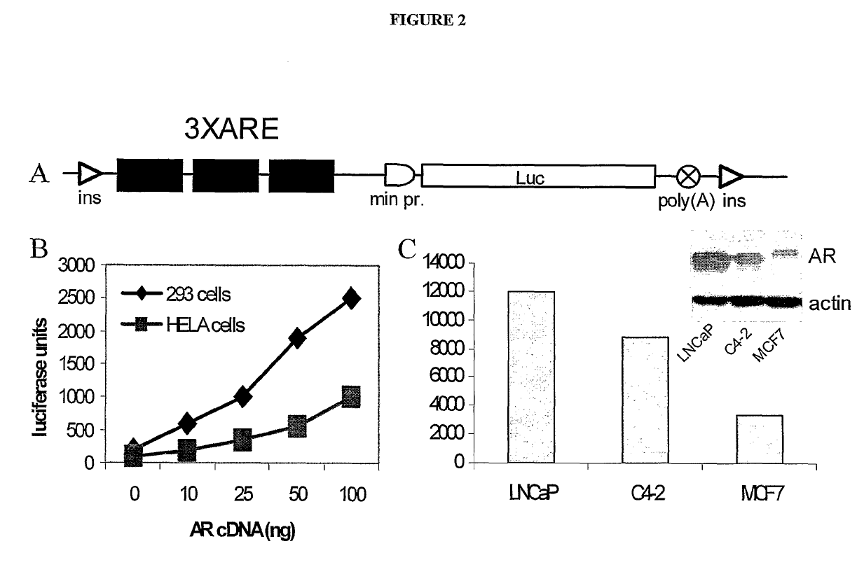

The ARELuc vector containing an AR reporter construct ARELuc (FIG. 2A) was prepared. Human AR, subcloned into pcDNA3Zeo vector was cut with BamH1 and Tth111I restriction enzymes. The fragment of AR corresponding to nucleotides 909-3095 of human AR (NM00041) was ligated with short oligonucleotide providing a stop codon through Tth111I cohesive ends and then cloned back into the pcDNA3.1Zeo vector using BamH1 and Xba1 sites. This fragment was also subcloned into lentiviral vector pLV.

The promoter contained a cassette of three androgen responsive elements (ARE) derived from the promoter of the rat probasin gene. The promoter also contained the Hsp70 minimal promoter, which by itself showed barely detectable background expression in prostate cell lines (data not shown). The gene encoding luciferase was used as a reporter gene under the control of the promoter. The reporter construct was flanked by two insulator sequences and includes a selectable marker for neo-resistance.

Cotransfection experiments with ARELuc demonstrated that reporter expression was low in the absence of AR using cells with low or zero levels of AR expression, but increased in a dose-dependent manner upon cotransfection of AR cDNA (FIG. 2B). In cells expressing endogenous AR, such as MCF7 (breast cancer), LNCaP and C4-2, basal activity of the reporter was detectable and proportional to the level of AR protein (FIG. 2C).

### Example 2

### Evaluation of LnCaP, C4-2 and CWR22R

We tested the AR reporter construct in three prostate cancer cell lines: LNCaP, C4-2 and CWR22R. Androgen-dependent LNCaP cells and their derivative C4-2 are derived from a xenograft of LNCaP cells grown in castrated animals. CWR22 cells are grown as a xenograft in mice, while their androgen-independent derivative CWR22R may be grown in culture. The AR gene in LNCaP-C4-2 pair has the same mutation in the ligand-binding domain (threonine→alanine at residue 877), which is frequently found in prostate cancer patients. CWR22 cells have another hot spot codon mutated (histidine→tyrosine at residue 874), while CWR22R cells acquired additional mutations (Leu→Gln at residue 57, Glu→Asp at residue 635), and a duplication of the third exon.

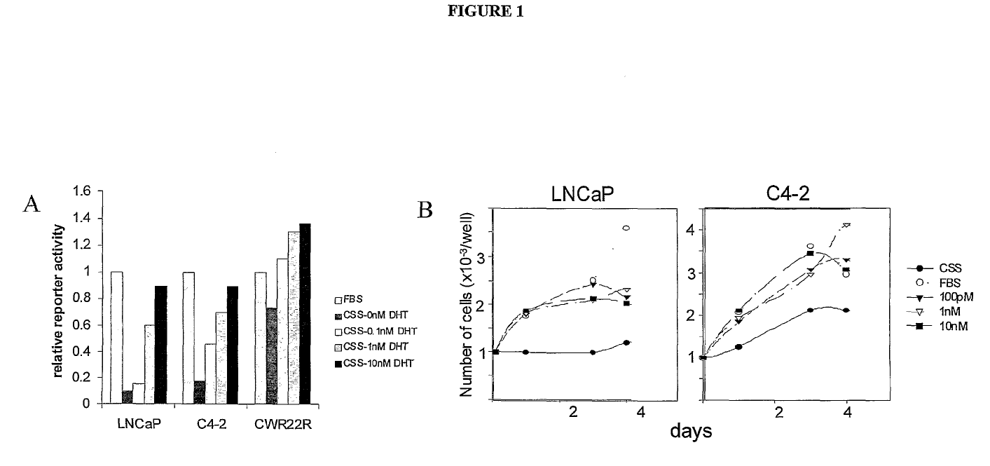

We transduced each cell type with the ARE-Luc reporter and tested the dependence of reporter activity on the presence of androgens and other ligands present in fetal bovine serum (FBS). We plated the cells in medium, supplied with charcoal stripped serum (CSS) containing 0-10 nm of dehydrotestosteron (DHT) as well as regular FBS. As shown in FIG. 1, all of the cells had reduced reporter activity and proliferation rate in CSS with no DHT addition. Each of the cells also responded to the addition of DHT by reporter activation and increased proliferation in a dose-dependent manner. This indicates that to differing degrees LNCaP, C4-2 and CWR22R are each androgen dependent. This was surprising because CWR22R cells are claimed to be androgen-independent.

### Example 3

### Ligand Independent Mutant of AR

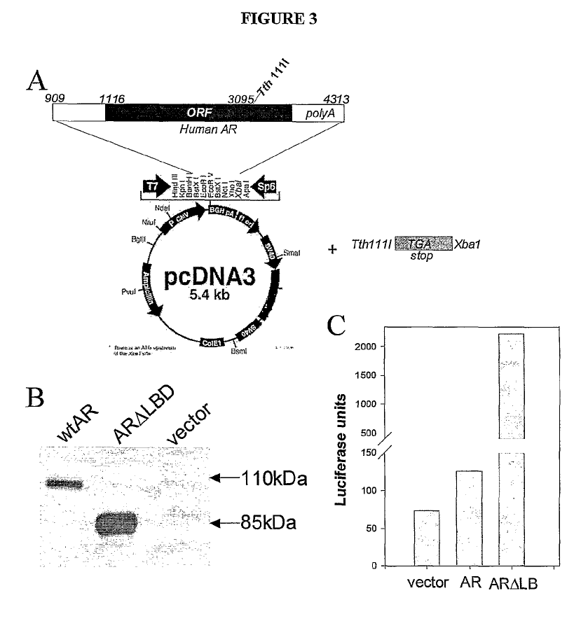

We sought to produce a more completely androgen-independent prostate cancer cell line by introducing a rationally designed AR mutant that would be more completely ligand independent. We chose to generate prostate tumor-derived AR mutants completely lacking the ligand-binding domain (LBD). We generated a truncated mutant (AA 1-659) and cloned it into pcDNA3zeo and lentiviral plasmid pLV (FIG. 3A). Expression of the ARΔLBD mutant was confirmed by a Western blot analysis of cell extracts after transfection of ARΔLBD as well as wild type AR into HeLa cells. We visualized ARΔLBD using antibodies targeted to the N-terminus of AR (85 kDa band, FIG. 3B). We tested the transactivation function of ARΔLBD mutant by cotransfection of this mutant together with the ARE-Luc plasmid into AR negative HeLa cells. As shown in FIG. 3C, the AR mutant was more than 10× active in the absence of any steroids compared to wtAR.

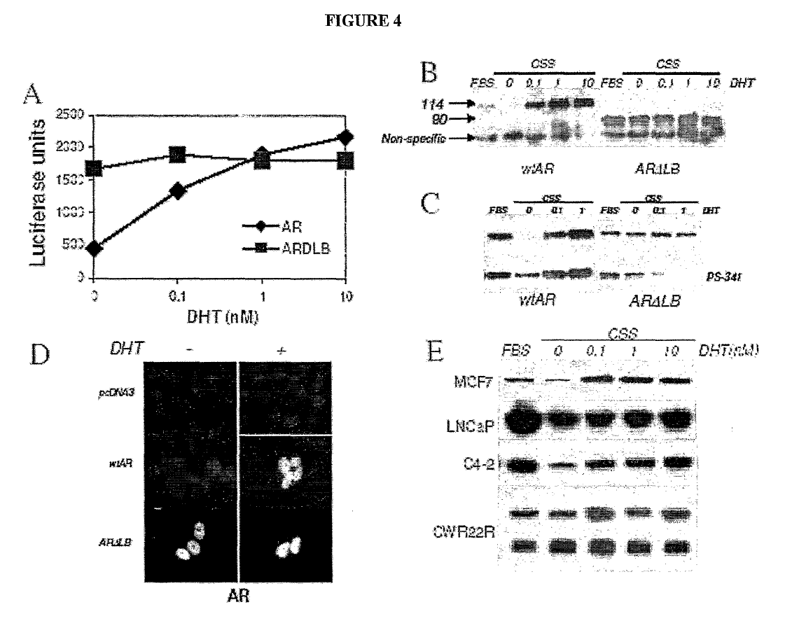

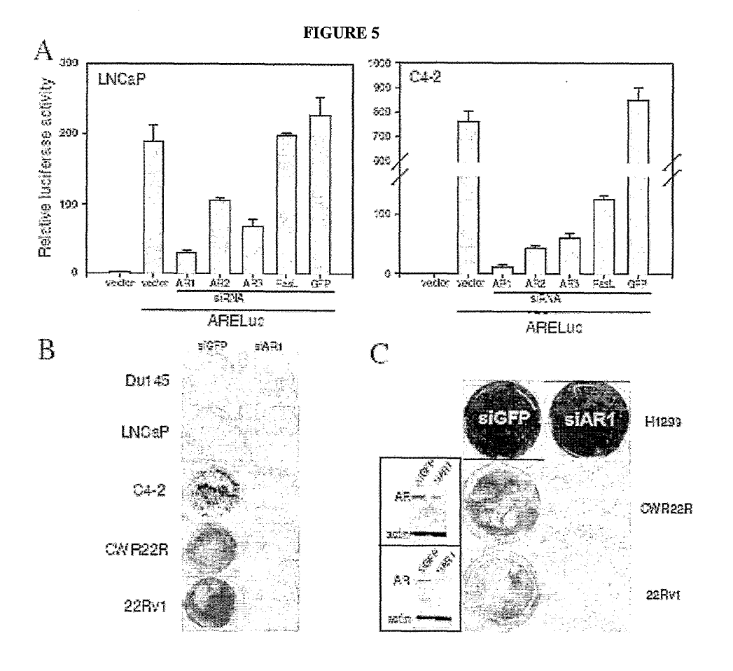

Dose-response analysis of ARΔLBD and wtAR in the presence of differing concentrations of DHT indicated that reporter activity in HeLa-wtAR cells was low in the absence of DHT and dose dependently stimulated by DHT, while luciferase activity in HeLa-ARΔLBD cells was always high (FIG. 4A). Moreover, the expression level of ARΔLBD was not dependent on DHT, while the level of wtAR was decreased in the absence of DHT (FIG. 4B). This indicates that the level of wild type AR protein is dependent on the level of DHT in the medium, while the level of the ARΔLBD mutant was independent of DHT. Since both proteins were expressed from the same viral promoter (CMV), this suggests that the LBD regulates the stability of wild type AR.

To confirm that the differences in the level of wtAR and ARΔLBD in the absence of DHT was due to the destabilizing effect of LBD and not to the different expression levels, we treated both variants of HeLa cells with PS341, which is an inhibitor of proteasomes. FIG. 4C indicates that wtAR levels increased in the presence of PS341, while the level of the ARΔLBD protein remained unchanged. Although it has been reported that wtAR is more stable in the presence of DHT, it was unknown that the stability of AR is determined by the LBD.

To confirm the role of the LBD on the stability of wtAR, we performed immunofluorescent staining of both variants of HeLa cells in the presence and absence of DHT. ARΔLBD was highly expressed regardless of DHT presence in the medium and was always localized exclusively in the nuclei (FIG. 4D), while wtAR was undetectable in the cells kept in a CSS medium for 24 hours and detectable in the presence of physiological concentrations of DHT. Importantly, wtAR was localized exclusively in the nuclei (FIG. 4D). This is unexpected since it was believed that wtAR is a cytoplasmic protein that undergoes nuclear translocation following DHT stimulation. Our results demonstrate that there is another level of AR regulation by ligands, since DHT binding leads to stabilization and nuclear translocation of wtAR.

The DHT-mediated stability of AR has until now been unappreciated. This may be due to the unavailability of normal prostate cell lines that express wtAR, since normal prostate epithelia grown in vitro originate from basal layer epithelial cells that do not express AR. In addition, available prostate tumor cell lines have mutations in the ligand-binding domain that perhaps lead to stabilization of AR. To confirm this, we monitored AR protein levels in different prostate cancer cell lines with different concentrations of DHT and found that the dependence of AR levels on DHT was much less prominent than in wtAR in HeLa cells (FIG. 4E). Mutant forms of AR in prostate cancer may be selected for higher protein stability, which may make their stability less ligand dependent.

### Example 4

### Elimination of Endogenous Ligand-Dependent AR Expression in Prostate Cancer Cells

To block residual AR activity, we generated several siRNA against AR (Table 2) and showed that their expression leads to suppression of the growth of both androgen-dependent and independent prostate cancer cells. We confirmed that inhibition of AR signaling is growth suppressive for androgen-insensitive versions of CaP (FIG. 5). Based on what is known from clinical oncology and AR mutation databases, we hypothesized that complete inhibition of AR function would be deleterious for both androgen-dependent and androgen refractory prostate cancer cells since CaP cells very rarely loose AR signaling during progression. To estimate the dependency of prostate cancer cells on the expression of AR, we transduced them with retroviral vector expressing anti-AR siRNA with subsequent selection on hygromycin. Control cells were transduced with anti-GFP siRNA vector. Transduction efficiency was estimated in AR-negative cells, DU145. AR1 siRNA inhibited the growth of LNCaP and C4-2 cells (FIG. 5B).

TABLE 2
siRNAs

| SiRNA | Sequence | SEQ ID NO | Target |
|---|---|---|---|
| AR1 | GCTCAAGGATGGAAGTGCA | 3 | 1108-1126 |
| AR2 | GCTGCTCCGCTGACCTTAA | 4 | 1642-1660 |
| AR3 | TCTCTGTGCAAGTGCCCAA | 5 | 3814-3832 |
| AR6 | GTCAGGTCTTCAGTAGCCA | 6 | 688-706 |

We also used methylene blue cell staining to evaluate growth inhibition of CaP cells 96 hrs after transduction with lentivirus expressing shRNA against AR (FIG. 5C). Control cells were transduced with siRNA to E6 HPV viral protein, absent from CaP cells. A partial decrease in AR expression achieved with less potent siAR constructs is easily circumvented by CaP cells without effect on their growth. These results confirmed cytotoxicity of AR inhibition for both androgen-dependent and independent cells (FIG. 5C).

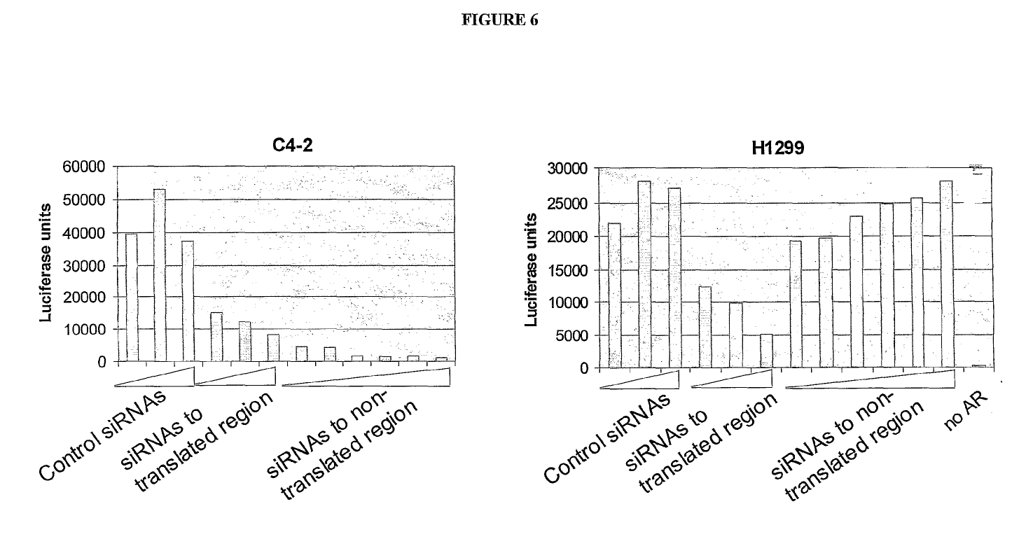

We next eliminated expression of the endogenous ligand-dependent AR in a way that would not affect the expression of the ARΔLBD mutant. To achieve this, we generated a series of lentiviral vectors expressing siRNA's against untranslated regions of AR mRNA that are missing in the generated ARΔLBD-expressing constructs. We tested their activity in C4-2 prostate cancer cells that have endogenous AR and in H1299 cells expressing only exogenous AR from the transduced construct. While all siRNA's against translated and untranslated regions of AR were active in inhibition of ARE-Luc activity in C4-2 cells, only siRNA's against translated regions were active in H1299 cells (FIG. 6). This demonstrates that expression of endogenous and exogenous AR may be separately modulated. The most effective siAR-6 was used for further study.

### Example 5

### Production of Ligand-Independent Prostate Cancer Cells

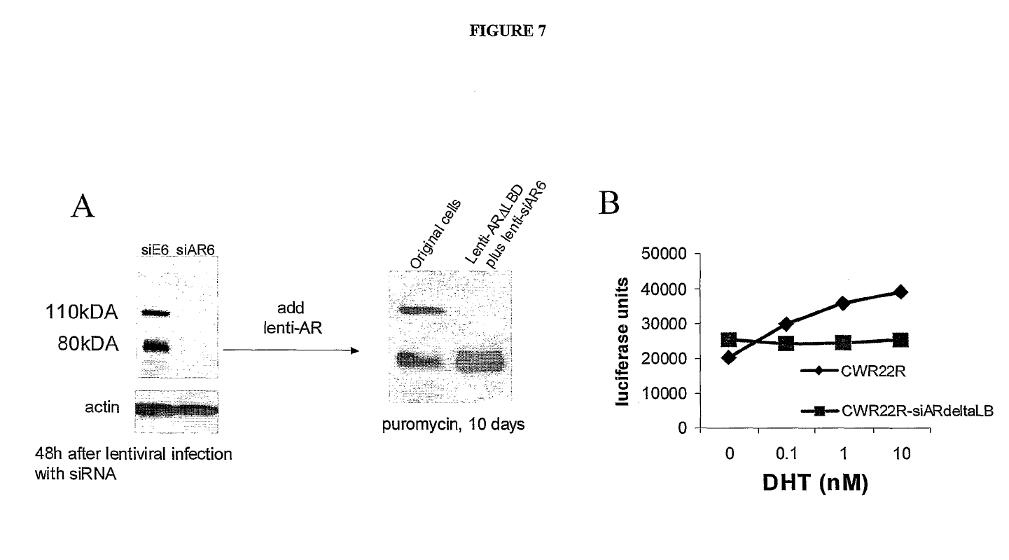

We next transduced prostate cancer cells with the ARΔLBD-expressing vector. We were unable to select prostate cancer cells overexpressing either ARΔLBD protein or wtAR, which was used as a control. Of the few colonies of cells that survived selection, none of the cells showed overexpression of wild type or mutant AR (FIGS. 7A and B). This indicated that increased activity of AR signaling also interferes with prostate cancer cell growth. In this regard, it is important to note that high doses of androgens are growth suppressive for prostate cancer cells and that normal prostate AR expression coincides with growth arrest and cell differentiation. Thus, prostate cancer cells may tolerate only a limited level of AR signaling. This may be due to complete loss of AR signaling leading to apoptosis and over-stimulation leading to growth arrest.

To generate completely ligand independent CaP cells, we simultaneously infected cells with lentiviral vectors expressing siAR6 and ARΔLBD. We were able to generate cells with nearly complete knock down of endogenous AR and visible expression of ARΔLBD (FIG. 7).

### Example 6

### CaP Cells Do Not Tolerate Overexpression of AR

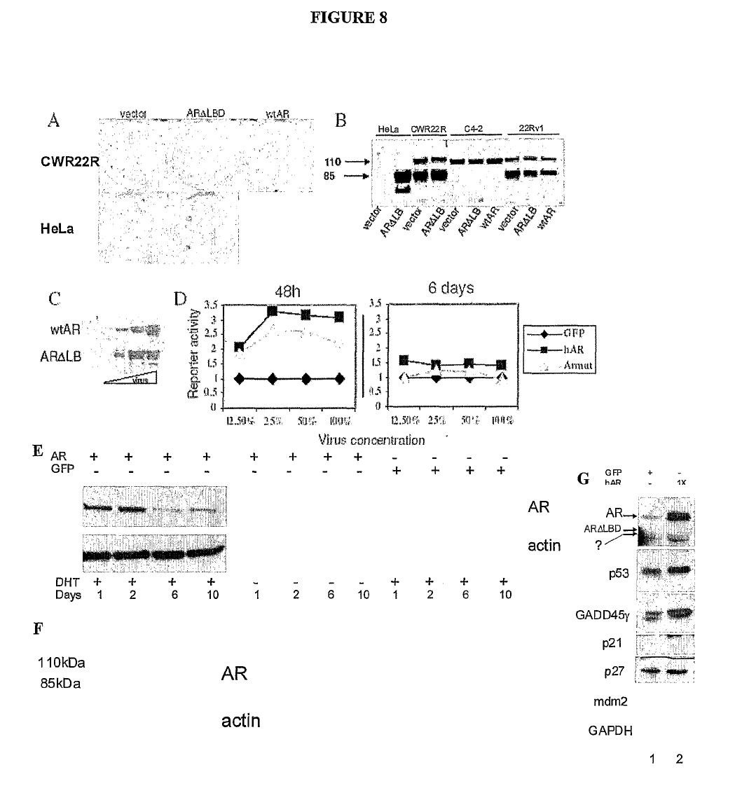

It has been reported that it is difficult to overexpress AR from strong promoters in CaP cells. We also observed that the number of clones selected after transfection of several CaP cell lines with wtAR, or ARΔLBD are much lower compared to empty vector. The rare CaP cell clones grown after transfection with AR variants in fact did not overexpress AR or ARΔLBD. We recloned AR cDNAs into lentiviral constructs and transduced them into prostate cancer cells along with control empty viruses. Overexpression of AR on Western blot as well as activation of ARELuc reporter was easily detectable at 48 hours after transduction (FIG. 8). However, AR protein level and ARELuc activity decreased to levels found in untransduced cells within 6 days after transduction.

The inability to get AR overexpressing clones as well as a decrease of exogenous AR level in the time during which AR overexpressing cells are most likely being eliminated from the cell population. This elimination did not happen if the cells were maintained in DHT-free medium, indicating that the observed intolerance to high AR receptor levels is associated with its activity.

To elucidate the mechanism underlying this effect we looked at the effect of different growth-inhibitory factors in cells transduced with AR using HT1080 cells. We observed an increase in expression of growth inhibitory proteins such as p21/Waf, p27/Kip proteins, and Gadd45γ. Since two of these growth inhibitors are direct targets of p53, we analyzed p53 protein level, which appeared induced as well as another p53 target, mdm2. Thus, modulation of AR expression either below or above tolerable levels results in similar changes in cell behavior: suppression of cell growth potentially through or at least with involvement of the p53 pathway.

### Example 7

### Growth of Prostate Cancer Cells In Vitro Depends on Transcriptional Activity of Androgen Receptor

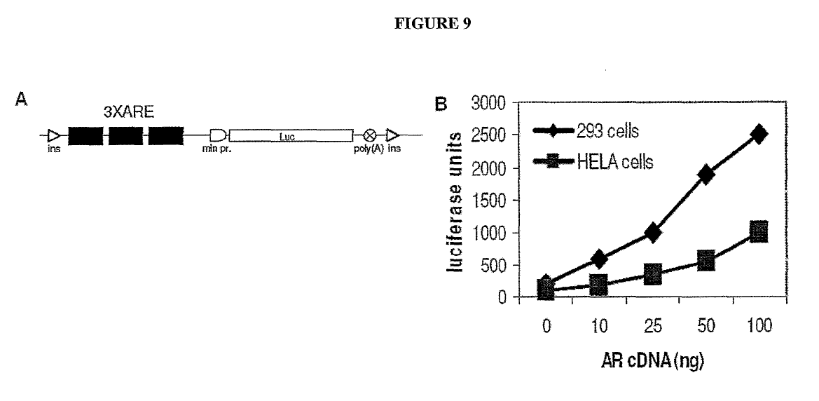

To monitor AR activity of cells we transduced them with an AR-responsive reporter (see FIG. 9A) and then selected for a population of cells with an integrated AR-dependent luciferase using G418. Adequate activity of the reporter was confirmed by experiments with androgens in AR expressing cells (see below) and with transfection of different doses of AR cDNA into AR-negative cells (FIG. 9B). Cells were maintained in DMEM with 10% FBS and antibiotics. pARE-Luc plasmid was constructed as shown in FIG. 9. Transfection was done using Lipofectamine Plus reagent (Invitrogen) according to manufacture's recommendations. 293 and Hela cells were transfected with 0.5 μg of pARE-Luc DNA and increasing amounts of an AR cDNA. Luciferase activity was measured in cell lystates 48 h after transfection. Reporter activity was measured by using a Luciferase Assay System (Promega) and normalization was done by protein content (DC Protein Assay, BioRad). 293 and Hela cells were obtained from ATCC and were maintained in DMEM with 10% FBS and antibiotics.

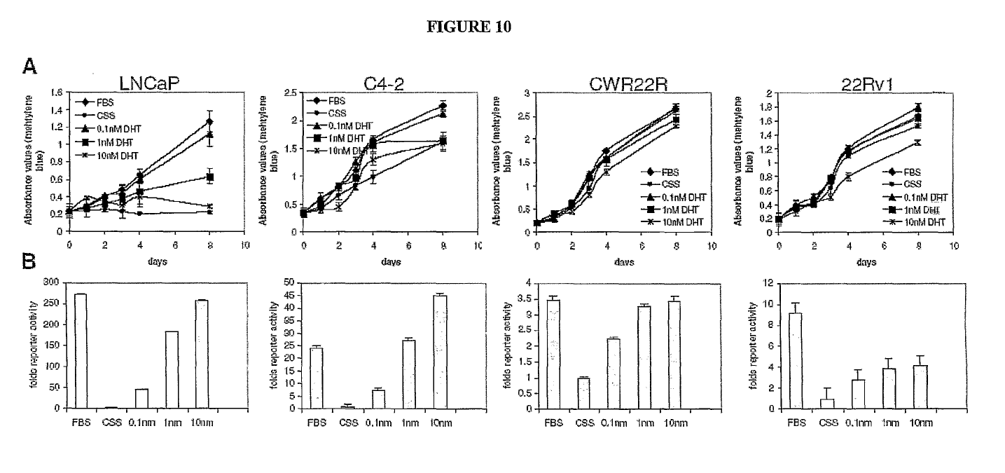

The comparison of cell dependence on androgens was done by monitoring cell growth (number of cells by methylene blue staining) and AR reporter activity in the medium, supplied with charcoal stripped serum (CSS) containing 0-10 nm of dehydrotestosterone (DHT)(FIGS. 10A and 10B).

Cells were plated in 12 well plates at 2×10⁴/well in duplicates. The next day, the medium was removed and cells were washed with PBS. Phenol-free RPMI-1640 with either FBS or CSS was added, plus all standard additives. After attachment, media in wells were changed with media with CSS and different concentrations of DHT, and this medium was changed every 48 h. Two plates per cell type were either fixed at the number of days indicated in FIG. 10 and stained with 0.5 μg/ml of methylene blue in 50% methanol solution or lysed with Cell Culture Reporter Lysis Reagent (Promega) and used in a Luciferase Reporter Assay (Promega). Methylene blue staining was quantified by extraction with 1% SDS in PBS solution and absorbance values were measured at 1=600. Luciferase readings were normalized by protein content (DC Protein Assay, BioRad). Three independent experiments were performed. Quantification was done by elution of staining with 1% SDS solution and spectrophotometry at λ=600 nm. Reporter activity in CSS medium without DHT (CSS) was taken as 1. For these experiments, LNCaP and 22Rv1 cells were obtained from ATCC, and C4-2 and CWR22R cells were provided by Warren Heston (Dept.of Cancer Biology, Cleveland Clinic Foundation). All prostate cancer cells were maintained in RPMI 1640 medium, supplemented with 10% FBS, 1 mM sodium pyruvate, 10 mM Hepes buffer, 55 nM B-mercaptoethanol and antibiotics. CSS medium was phenol red-free and used with the same additives as mentioned above. DHT (dehydrotestosterone) was obtained from the Cleveland Clinic Foundation Pharmacology Department.

The experiments shown in FIGS. 10A and 10B demonstrated that LNCaP cells stop proliferating in CSS in parallel with decreases in AR activity in the absence of DHT. DHT-stimulated proliferation of LNCaP cells and induced AR-dependent reporter activity up to more than 250 times. Importantly, while DHT stimulated transcription in a dose-dependent manner (i.e., the higher the dose of DHT, the higher the AR-dependent reporter activity), proliferation of LNCaP cells was stimulated only by low physiological doses of DHT (0.1-1 nM). Higher levels of DHT did not stimulate proliferation of LNCaP cells.

Androgen-insensitive C4-2 cells did not stop proliferating, but their growth in CSS medium was reduced, although not as much as compared to LNCaP cells (FIGS. 10A and B). DHT minimally stimulated proliferation of C4-2 cells, but induced AR-dependent reporter activity, although less so than in the case of LNCaP cells (up to 45 times). Both CWR22R and 22Rv1 cells reacted minimally to a DHT deficit in terms of proliferation, although reporter activity decreased in the absence of DHT. The decrease was several times lower than in LNCaP or C4-2 cells. DHT did not stimulate proliferation of CWR22R and 22Rv1 cells, and reporter induction was minor compared to that of LNCaP and C4-2 cells (up to 3.5 fold). Although physiological levels of DHT did not affect proliferation of CWR22R cells, high doses of DHT (10 nM) had a minor growth suppressing effect (FIGS. 10A and B). Growth of AR-negative Hela cells was not affected by DHT (data not shown), indicating that AR is primarily target of DHT and modulation of AR activity accounts for the different growth properties of CaP cells.

These experiments demonstrated that correlation between AR transcription and proliferation of CaP cells was not linear. First, changes in AR transcriptional activity in all cells, AS and AI, were several times more significant than changes in cell proliferation (e.g., in LNCaP cells in which a 50- to 272-fold change in AR-dependent reporter activity and a 2- to 6-fold change in proliferation were observed). Second, androgen-independence of cells does not mean complete independence of AR transcription from the presence of hormones. That is, the absence of DHT leads not to silencing of AR signaling, but to some decrease in AR-dependent transcription. The degree of this decrease correlated well with the proliferative response of CaP cells. The largest decrease (more than 250-fold) occurred in the most AS-type of cells (LNCaP), an intermediate decrease (23-fold) occurred in partially-AI cells (C4-2), and a minimal decrease (3.5- to 9-fold) occurred in the most AI-type of cells (CWR22R and 22Rv1). Based on these data we concluded that the androgen independence of CaP cells indicated constitutive transcriptional activity of AR with a minor response to ligands.

### Example 8

### Effect of AR Knockdown on Growth of Androgen-Dependent and -Independent CaP Cells

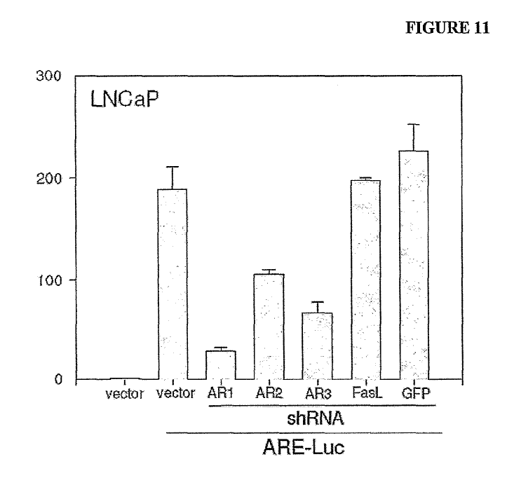

Based on what was known from clinical oncology and AR mutation databases we hypothesized that complete inhibition of AR function would be toxic for both androgen-dependent and androgen-refractory prostate cancer cells, because prostate cancer cells very rarely lose AR signaling during cancer progression. To completely block AR activity in these cells we used an RNAi approach (FIG. 11). We synthesized several shRNA constructs targeting different portions of AR mRNA using a loop model (see Example 4 above). To chose the most active shRNA constructs from synthesized pool we first tested effect of these shRNAs in cotransfection experiments with an AR-responsive reporter along with the control vector expressing shRNA to GFP (shRNA to FasL was used as an additional control) in cells with endogenous AR (LNCaP) assuming that loss of AR expression would lead to drop in reporter activity.

Reporter assays were performed by transiently transfecting cells with pARE-Luc plasmid and pcDNA-3.1 hygro plasmids (empty or AR), or plasmids with shRNAs in different proportions. Transfection efficiency was normalized by citransfection of pCMV-LacZ plasmid or pEGFP-mito (Clontech). Reporter activity was measured using a Luciferase Assay System (Promega) at 48 h.

All three AR-specific shRNA suppressed to some degree activity of the reporter on endogenous AR in LNCaP cells. But the degree of inhibition was different: maximal activity was demonstrated by shAR1 in all systems, two others were less effective (FIG. 11).

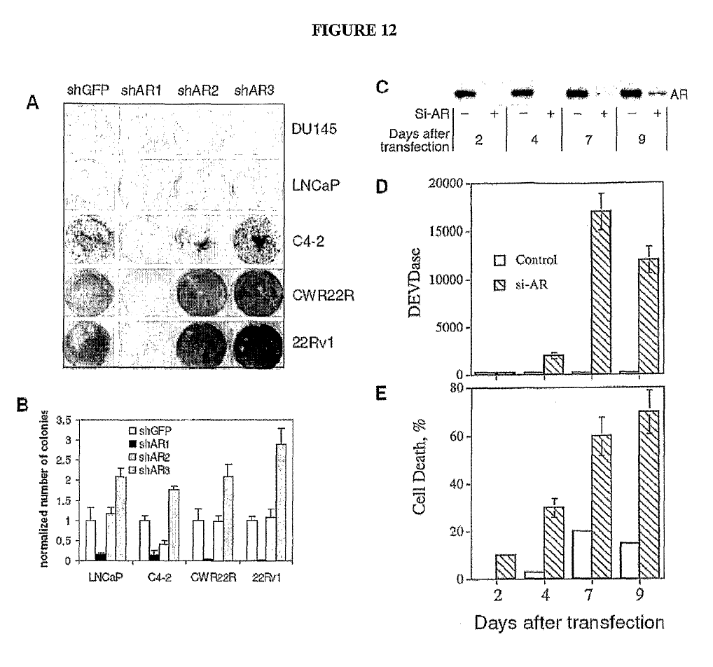

We also estimated the dependency of prostate cancer cells on the expression of AR by transducing cells with a retroviral vector expressing anti-AR shRNA, followed by selection on hygromycin (FIG. 12). Control cells were transduced with anti-GFP shRNA vector; transduction efficiency was estimated on AR negative cells (DU145) transduced with the same viruses.

For the experiments in FIG. 12, retroviral packaging and transduction were performed as follows. Briefly, Ampho cells (Clontech) were transfected with retroviral expression vector. Medium with virus was collected at 48 h and immediately transferred onto the target cells. 8 μg/ml of polybrene (Sigma) was added to transduced cells. 24 h later, the medium was changed with fresh medium containing the appropriate antibiotic for selecting transduced cells. 10-14 days later, after complete death of control untransduced cells, the number of colonies was quantified, or cells were used for further experiments. Retroviral shRNA vectors were generated by inserting the H1 promoter and a cassette for cloning of shRNA into the right LTR of pLPCHygro plasmid (Clontech).

For the experiment in FIG. 12B, the number of colonies on each plate was counted and divided by number of colonies of AR-negative DU145 cells. The number of colonies of cells transduced with shRNA against GFP was set as 1. DU145 cells were obtained from ATCC.

For the experiment in FIG. 12C, cells were lysed in Cell Culture Reporter Lysis Reagent (Promega). Protein concentrations were determined with DC Protein Assay (BioRad). Equal protein amounts were run on gradient 4-20% precast gels (Novex) and blotted onto PVDF membranes (Amersham). anti-pAR—monoclonal mouse antibody (Pharmigen, BD) was used. HRP-conjugated secondary antibodies were purchased from Santa-Cruz. Quantification of the data was performed using Quantity One software from BioRad.

These experiments demonstrated the growth inhibitory effect of only AR1 shRNA, the most effective in inhibition of reporter activity, on the growth of all tested CaP cells expressing endogenous AR (FIGS. 12A and B) and no inhibitory activity of shAR2 (with exception of C4-2) and shAR3 constructs, which did not completely block AR transcription. Moreover, the shAR3 construct consistently demonstrated some growth-inducing property on AR expressing CaP cells, which may indicate that partial suppression of AR expression and activity has a growth-promoting effect. Thus, significant inhibition of AR expression in CaP cells was toxic for these cells, independent from the cells' androgen sensitivity. A partial decrease in AR expression, achieved with less potent shAR constructs, was easily circumvented by CaP cells, without an inhibitory effect on their growth.

To trace the fate of cells with inhibited AR expression we transfected cells with siRNA oligos specific to human AR (100% transfection efficiency as estimated by fluorescent labeling of oligos). Caspase activation and death of CaP cells with inhibited expression of AR by siRNA was slow. Expression was noticeable 4 days after transfection and significantly increased at 7 and 9 days post-transfection (FIGS. 12D and E). AR expression decreased much more quickly (48 hours after transfection, FIG. 12C). The delayed effect of knock-down of AR expression on cell survival may be explained by the fact that it is mediated by an AR dependent factor with prolonged RNA or protein half-life.

These results indicated a critical role for AR signaling in the growth of prostate cancer cells, independent of the stage of tumor progression.

### Example 9

### Overexpression of AR

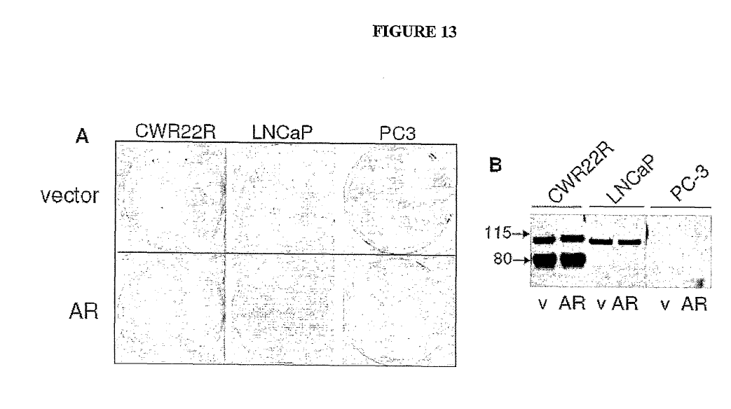

To determine whether AR overexpression would facilitate cell growth or protect from cell death, we expressed AR in cells that express different levels of endogenous AR. These cells were: LNCaP, C4-2 and CWR22R, and CaP cells that have lost AR expression (i.e., PC3 cells). We selected cells on hygromycin and after normalization of transfection efficiency, and compared the number of clones. In each case, expression of AR caused a significant reduction in the number of clones comparing with empty vector (FIG. 13A). Those rare clones which were selected did not express or overexpress AR (FIG. 13B).

Transfection efficiency was normalized by cotransfection with pCMV-β-gal. Some colonies that survived hygromycin selection were expanded and the amount of AR protein was estimated by Western blotting. The AR cDNA was provided by AO Brinkmann (Department of Biochemistry, Erasmus University, Rotterdam, The Netherlands) and cloned into pcDNA3.1hygro plasmid (Invitrogen).

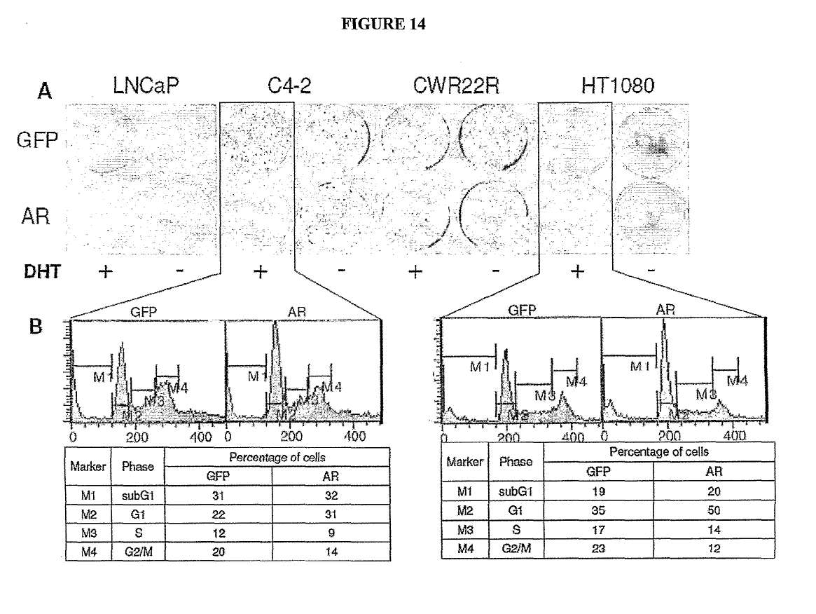

Similar results to those in FIG. 13 were obtained when we transduced cells with lentiviral vectors expressing AR or GFP as a control (100% transduction efficiency based on GFP fluorescence)(FIG. 14).

Lentiviral packaging and transduction were performed as follows. Briefly, 293 cells were transfected with equal amounts of lentiviral-expressing vector, packaging plasmid pLV-CMV-delta 8.2 (provided by Inder Verma, Salk Institute, Calif.) and pVSV-G plasmid (Clontech) for pseudotyping. Virus-containing medium was collected at 48 and 96 hours and pooled. In some cases virus was concentrated 20 times by incubation of medium from 293 cells overnight at 4° C. in the presence of 40% PEG8000 followed by centrifugation at 6000 rpm. The protein pellet and virus were then dissolved in the appropriate medium and stored at −80° C. Target cells were transduced by incubating in virus-containing medium for 24 h. Viral titer was detected either by GFP fluorescence (GFP virus) or by transduction of AR-negative Hela cells (AR virus) followed by immunofluorescent staining with anti-AR antibodies (at 48 h after transduction).

Cells were plated in 6 well plates at 10⁵/well. The next day, cells were transduced with concentrated GFP or AR lentiviruses, yielding 50-100% transduction efficiency. 24 h later, the medium with virus was removed, and cells were washed with PBS followed by phenol-free RPMI-1640 with CSS, with or without 0.3 nM DHT. The medium was changed every 48 h. Cells were collected for Western blotting and luciferase assays at different time points or fixed and stained with methylene blue on day 8 after transduction.

Around 10⁵ cells were trypsinized according to standard protocol. The cell pellet was washed with PBS and resuspended in 300 μL of 3% BSA in PBS and then 5 ml of 70% ethanol added in drops. Cells were kept at −20° C. for several hours and then stained with 10 μg/ml of PI in the presence of 30 μg/ml of RNAsa A at 37° C. for 2 hours. Cell cycle distribution was then analyzed using FACS Calibur (Becton Dickinson) and CellQuest software.

At 10 days after transduction, colonies of cells expressing AR were significantly smaller compared to those expressing a GFP control (FIG. 14A). This indicated that the increased level of AR lead to growth suppression even in cells expressing endogenous active AR. Moreover, AR overexpression also suppressed growth in fibrosarcoma HT1080 cells, which express low levels of endogenous AR. Analysis of cell cycle distribution of cells overexpressing AR revealed a higher proportion of cells in G1 phase of cell cycle and lower proportion of cells in S and G2/M phases, again demonstrating the decrease of proliferation pool in cell populating overexpressing AR (FIG. 14B).

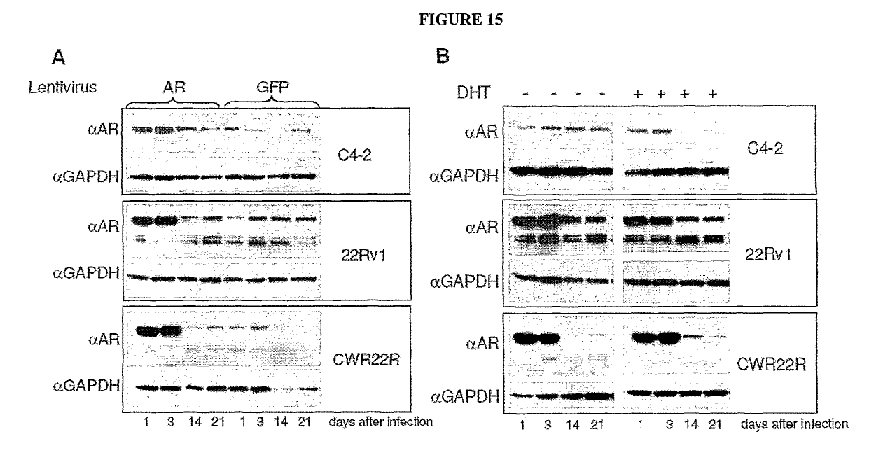

To ensure that cells overexpressing AR were lost in the process of selection we transduced cells with lentivirus with AR in a dose infecting around 50% of cells in the population. These cells were then propagated for two weeks in culture, and aliquots of cells were taken for Western blotting. Results of this experiment demonstrate that AR was overexpressed after transduction, but expression of AR gradually decreased to the level of untransduced cells. Importantly, the proportion of GFP positive cells after transduction with control GFP-containing virus was not changed (FIG. 15A and data not shown).

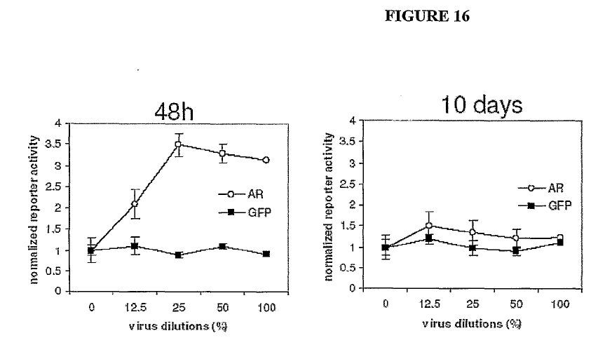

We measured AR-dependent transactivation by AR-dependent reporter activity in cells transduced with lentiviral AR. The transcription activity of AR, after an initial significant stimulation, also gradually decreased to the level of untransduced cells (FIG. 16).

To determine whether AR-dependent transactivation is important for the inhibitory effect of AR on cell growth, we cultured half of transduced cells in CSS-containing media, lacking steroids and the other half on medium with 0.3 nM of DHT. The growth of cells overexpressing AR, cultured in CSS with no DHT, was not different from GFP expressing cells. Thus, this growth reflects the general sensitivity of certain type of cells to the presence of steroids in medium (FIG. 13A). The presence of 0.3 nM of DHT leads to suppression of growth of AR overexpressing cells compared to a GFP expressing control. The level of AR proteins in cells transduced with AR-lentivirus and cultured in CSS medium without DHT did not decrease. However adding DHT-containing media reduced AR levels to those observed prior transduction (FIG. 15B). Importantly, AR levels did not decrease in the absence of steroids, except in cells in which AR transcription significantly depended on ligands (e.g., C4-2 cells). In cells in which transcriptional function of endogenous AR was not ligand-dependent (e.g., CWR22R and 22Rv1 cells), the level of exogenous AR protein decreased, even in the absence of DHT, most likely due to a sufficient level of endogenous and exogenous AR transcriptional activity. Thus, CaP cells do not tolerate overexpression of AR.

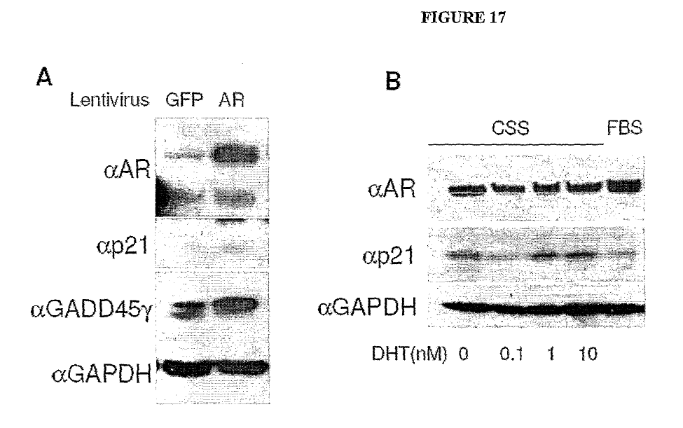

We also looked at the level of p21 in cells transduced with AR. p21 is a CDK inhibitor that is expressed in many tumor cells, and p21 protects cells from apoptosis. The effect of p21 can be overcome by overexpressing cyclin D. Inducing p21 leads to an imbalance of cyclin D and p21, resulting in growth arrest and elimination of p21-expressing cells from the pool of proliferating cells. We infected CWR22R cells with a lentiviral construct containing either AR or GFP as a control and 48 h after transduction prepared lysates of the cells. The level of p21 protein was elevated in cells transduced with AR lentivirus, comparing with cells transduced with GFP (FIG. 17A). In addition, expression of the growth suppressor GADD45g increased. The following antibodies were used: anti-p21—monoclonal mouse F-5 antibody (Santa-Cruz), and anti-GADD45g—mouse monoclonal antibody (Santa-Cruz)

To ensure that p21 level was not induced solely due to elevated levels of AR protein, but also because higher levels of AR transcription, we activated AR in LNCaP cells using different concentrations of DHT. As shown on FIG. 17B, the level of p21 was higher in lysates of cells treated with 1 nM and 10 nM of DHT, compared to cells cultured in 0 nM or 0.1 nm of DHT or in medium with FBS (roughly equivalent to 0.3 nM level of DHT). Importantly, LNCaP cells maintained at 10 nM DHT proliferate more slowly than cells cultured in lower levels of hormone (FIG. 10). Thus increased activity of AR due to its overexpression or hyper-stimulation with ligand inhibits cell growth, possibly due to induction of p21.

1. A target cell comprising a constitutively active mutant AR, wherein the target cell is substantially androgen-independent.

2. The target cell of claim 1, wherein the mutant AR is lacking the ligand binding domain of wtAR.

3. The target cell of claim 2, wherein the wtAR comprises the sequence of SEQ ID NO: 2 or a sequence at least 80% identical thereto.

4. The target cell of claim 3, wherein the mutant AR is encoded by residues 1116-3878 of SEQ ID NO: 1 or a sequence at least 80% identical thereto.

5. The target cell of claim 3, wherein the mutant AR is lacking the C-terminal 248 to 295 residues of SEQ ID NO: 2.

6. The target cell of claim 5, wherein the mutant AR is lacking the C-terminal 261 residues of SEQ ID NO: 2.

7. The target cell of claim 6, wherein the mutant AR comprises residues 1-659 of SEQ ID NO: 2 or a sequence at least 80% identical thereto.

8. The target cell of claim 1, wherein the target cell comprises an expression control sequence operatively linked to a reporter gene, wherein the expression control sequence comprises an ARE.

9. The target cell of claim 1, wherein the target cell comprises a second AR.

10. The target cell of claim 1, wherein the target cell comprises a siRNA comprising a sequence substantially complementary to a gene encoding the mutant AR.

11. The target cell of claim 9, wherein the target cell comprises a siRNA comprising a sequence substantially complementary to a gene encoding the second AR.

12. The target cell of claim 11, wherein the siRNA comprises a sequence that is not substantially complementary to a gene encoding the mutant AR.

13. A method for screening an agent for modulating AR activity:
(a) contacting the agent with the target cell of claim 1;
(b) determining the level of reporter produced by the cells in the presence and absence of the agent,
wherein a difference in the level of reporter compared to a control indicates that the agent is a modulator of AR activity.

14. A method of treating prostate cancer comprising administering to a patient in need thereof a composition comprising a modulator of AR signaling, wherein the modulator does not affect ligand binding.

15. The method of claim 14 wherein the prostate cancer is androgen refractory prostate cancer.


SEQUENCE LISTING (23 sequences)

SEQ ID NO: 1 - DNA, 4314 bp, Homo sapiens
cgagatcccggggagccagcttgctgggagagcgggacggtccggagcaagcccagaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaacaagcaaac

SEQ ID NO: 2 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 3 - DNA, 19 bp, Artificial
  siRNA
gctcaaggatggaagtgca

SEQ ID NO: 4 - DNA, 19 bp, Artificial
  siRNA
gctgctccgctgaccttaa

SEQ ID NO: 5 - DNA, 19 bp, Artificial
  siRNA
tctctgtgcaagtgcccaa

SEQ ID NO: 6 - DNA, 19 bp, Artificial
  siRNA
gtcaggtcttcagtagcca

SEQ ID NO: 7 - DNA, 15 bp, Artificial
  androgen response element
agaacagcaagtgct

SEQ ID NO: 8 - DNA, 15 bp, Artificial
  androgen response element
ggatcagggagtctc

SEQ ID NO: 9 - protein, 919 aa, Human
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 10 - protein, 902 aa, Rat [sequence omitted]

SEQ ID NO: 11 - protein, 899 aa, Mus musculus
MEVQLGLGRVYPRPPSKTYRGAFQNLFQSVREAIQNPGPRHPEAANIAPPGACLQQRQETSPRRRRRQQHTEDGSPQAHIRGPTGYLALEEEQQPSQQQAASEGHPESSCLPEPGAATAPGKGLPQQPPAPPDQDDSAAPSTLSLLGPTFPGLSSCSADIKDILNEAGTMQLLQQQQQQQQHQQQHQQHQQQQEVISEGSSARAREATGAPSSSKDSYLGGNSTISDSAKELCKAVSVSMGLGVEALEHLSPGEQLRGDCMYASLLGGPPAVRPTPCAPLPECKGLPLDEGPGKSTEETAEYSSFKGGYAKGLEGESLGCSGSSEAGSSGTLEIPSSLSLYKSGALDEAAAYQNRDYYNFPLALSGPPHPPPPTHPHARIKLENPLDYGSAWAAAAAQCRYGDLGSLHGGSVAGPSTGSPPATTSSSWHTLFTAEEGQLYGPGGGGGSSSPSDAGPVAPYGYTRPPQGLTSQESDYSASEVWYPGGVVNRVPYPSPNCVKSEMGPWMENYSGPYGDMRLDSTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGENSNAGSPTEDPSQKMTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 12 - protein, 895 aa, Rhesus monkey
MEVQLGLGRVYPRPPSKTYRGAFQNLFQSVREVIQNPGPRHPEAASAAPPGASLQQQQQQQQETSPRQQQQQQQGEDGSPQAHRRGPTGYLVLDEEQQPSQPQSAPECHPERGCVPEPGAAVAAGKGLPQQLPAPPDEDDSAAPSTLSLLGPTFPGLSSCSADLKDILSEASTMQLLQQQQQEAVSEGSSSGRAREASGAPTSSKDNYLEGTSTISDSAKELCKAVSVSMGLGVEALEHLSPGEQLRGDCMYAPVLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGAGEAGAVAPYGYTRPPQGLAGQEGDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETAQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 13 - protein, 912 aa, Crocuta crocuta
MEVQLGLGRVYPRPPSKTYRGAFQNLFQSVREVIQNPGPRHPEATSAAPPGARLQQQHQHQQQHQHETSPRRQQQQQPEDGSPQRPSRGPTSYLALDEEQQPSQHQSAKGHPESGCVPEPVAMSRTGKGLEQQQPAPPDEDDSAAPSTLSLLGPTFPGLSSCSTDLKDILSEAGTMQLLQRQRQRQQQRQQQQQQQQQQQQQQQQQQEVVSEGGSSGRAREAAGAPTSSKDSYLGGSSTISDSAKELCKAVSVSMGLGVEALEHLSPGEQLRGDCMYAPLLGGPPPVCPCAPLTECKGSVLDDGPSKGTEETAEYSPFKTGYAKGLDGDSLGCSGSSQAGGSGTLEIPSTLSLYKSGTLDEAAAYQSRDYYNFQLSLAGPPPPPPSPHPHARIKLENPLDYGSAWAAAAAQCRYGDLASLHGGGAAGPGSGSPSATASSSWHTLFTAEEGQLYGPCGGSGGGGTGESVSVTPYGYTRPQQGLTGQEGDFPPPDVWYPGGVVSRMPYPSASCVKSEMGPWMESYSGPYGDMRLETTRDHVLPIDYYFPPQKTCLICGDEASGCHYGALTCGSCKVFFKRAAEGKQKYLCASRNDCTIDKFRRKNCPPCRLRKCYEAGMTLGARRLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKDLDRIIACKRKNPTSCSRRFYQLTKLLDSVQPIARELHQFTFDLLIKSHMVSVDFPEMMAEIISVQVPKILSGKVKPIYFHTQ

SEQ ID NO: 14 - protein, 920 aa, Homo sapiens; identical to SEQ ID NO: 2

SEQ ID NO: 15 - protein, 911 aa, Pan troglodytes
MEVQLGLGRVYPRPPSKTYRGAFQNLFQSVREVIQNPGPRHPEAASAAPPGASLLLQQQQQQQQQQQQQQQQQQQQQQETSPRQQQQQGEDGSPQAHRRGPTGYLVLDEEQQPSQPQSAPECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDSAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGEAGAVAPYGYTRPPQGLAGQEGDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 16 - protein, 895 aa, Macaca mulatta; identical to SEQ ID NO: 12

SEQ ID NO: 17 - protein, 907 aa, Canis familiaris
MEVQLGLGRVYPRPPSKTYRGAFQNLFQSVREVIQNPGPRHPEAVSAAPPGAHLQQQQQQQQQQETSPRQQQQQQQGDDGSPQAQSRGPTGYLALDEEQQPSQQRSASKGHPESACVPEPGVTSATGKGLQQQQPAPPDENDSAAPSTLSLLGPTFPGLSSCSTDLKDILSEAGTMQLLQQQRQQQQQQQQQQQQQQQQQQQEVVSEGSSSGRAREAAGASTSSKDSYLGGSSTISDSAKELCKAVSVSMGLGVEALEHLSPGEQLRGDCMYAPLLGGPPAVRPCAPLAECKGSLLDDGPGKGTEETAEYSPFKAGYAKGLDGDSLGCSSSSEAGGSGTLEMPSTLSLYKSGALDEAAAYQSRDYYNFPLSLGGPPPHPPPPHPHTRIKLENPLDYGSAWAAAAAQCRYGDLASLHGAGAAGPSSGSPSATTSSSWHTLFTAEEGQLYGPCGGSGGGSAGDGGSVAPYGYTRPPQGLAGQEGDFPPPDVWYPGGVVSRVPFPSPSCVKSEMGSWMESYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASNVTSPTEEPTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 18 - protein, 899 aa, Mus musculus; identical to SEQ ID NO: 11

SEQ ID NO: 19 - protein, 902 aa, Rattus norvegicus [sequence omitted]

SEQ ID NO: 20 - protein, 895 aa, Sus scrofa
MEVQLGLGRVYPWPPSKTFRGAFQNLFQSVREVIQNPGPRHPEAASAAPPGARLQQQQLQQQETSPRRQQQQQQPSEDGSPQVQSRGPTGYLALDEKQQPSQQQSAPECHPESGCTPEPGAASAASKGLQQQPPAPPDEDDSAAPSTLSLLGPTFPGLSSCSTDLKDILSEAGTMQLLQQQQQQQQQQEAVSEGNSSGRAREATGAPISSKDSYLGGSSTISDSAKELCKAVSVSMGLGVEALEHLSPGEQLRGGCMYAPLLTGPPSVRPTPCAPLAECKGSLLDDGPGKSNEETAEYSPFKAGYTKGLDSESLGCSSGGEEGGSGTLELPSALSLYKSGALDDVAAYPSRDYYNFPLALARPPPPPPPPHPHARIKLENPLDYGSAWAAAAAQCRYGDLASLHGGGASGPGSGSPSATSSSSWHTLFTAEESQLYGPCGGGGGGSAGEAGAVAPYGYTRPPQGLAGQEGDLAIPDIWYPGGVVSRVPYPSPSCVKSEMGPWMESYSGPYGDMRLEPTRDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSATSPTEEPAQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIVMQEKNPTSCSRRFYQLTKLLDSVQPIARELHQFTFDLLIKSHMVSVDFPEMMAEIISVQVPKILSGKVKPIYFHTQ

SEQ ID NO: 21 - protein, 703 aa, Gallus gallus
MEVQLGIGRVYPRPPGRTFRGVFQTFFQSVCEAFQAPRDEPGPGLPAPGAPCPQSPRPPPVASPAFLPLPEPRAAARPAMGSPFPCAGDLKELLGEPGVLPLLPPEAEPGAGRAEPALKEDFLGDSAKELCKAVSASMGLAVETLEAPREPPPREDCMFALPGGPPRAPRPDAAEPPEPPTPAAFKGSGAEAALAVEVPAGLPLYRVSPPPEEPPGRDCFVLPPPARIKLESPPEPAAVGAWGSPVPAPPWPSFFADEGQLYGPCAEPPPGAFGCGRPENADFAADAWHPMARAPYAAPGSCIKSELGPWAEGYAGAYGDVRLEAGREHILPIDYYFPPQKTCLICGDEASGCHYGALTCGSCKVFFKRAAEGKQKYLCASRNDCTIDKFRRKNCPSCRLRKCYEAGMTLGARKLKKLGSLKTQDEAEAASSSSPTEEQAPKMVMTHVNGFECQPIFLNVLEAIEPAVVCAGHDNSQPDSFSNLLSSLNELGERQLVYVVKWAKALPGFRNLHVDDQMSIIQYSWMGLMIFAMGWRSFTNVNSRMLYFAPDLVFNEYRMHKSRMYSQCVRMRQLSQEFGWLQITPQEFLCMKALLFFSIIPVDGLKNQKLFDELRMNYIKELDRIIACKRKNPTSCSRRFYQLTKVLDSVHPIAKDLHQFTFDLLIKAHMVSVDYPEMMAEIISVQVPKILSGKVKPIYFHAE

SEQ ID NO: 22 - protein, 509 aa, Bos taurus
MEVQLGLGRVYPRPPSKTYRGAFQNLFQSVCEVIQNPLPRHPEASSAAPSGARLQQQQETSPRQQQQQQQQREDGSPQVQSRGPTGYLALEEEQQPSQHHSAPEGHPESGCVPEPRAASAAGKGLQQPPPAPLDEDDSAAPSTLSLLGPTFPGLSSCSTDLKDILSEAGTMQLLQQQQQQQQEAVSEGSSGRAREATGAPISSKDSYLGGSSTISDSAKELCKAVSVSMGLGVEALEHLSPGEQLRGDCMYAPLLGPPAAVRPTPCAPLAECKGSLLDDGPSKGTEETAEYSPFKAGYTKGLDTESLSCSGSGEAGGSGTLELPSTLSLYKSGALDEVAAYQTRDYYNFPLALAGPPPPPPPPHPHARIKLENPLDYGSAWVAAAAQCRYGDLASLHGGGAAGPGSGSPSAAASSSWHTLFTAEEGQLYGPAVCGGGGGGSAGEAGAVAPYGYTRPPQGLAGQEGDFSPPDVWYPGGVVSRVPYPSPSCVKSEMGPWMENYSGPYGDMR

SEQ ID NO: 23 - protein, 413 aa, Danio rerio
MYDGGLKMQRGVDHNGLSPMKNGSRKWFMLLFLFSRFDGGRSDIFPMEFFLPPQRTCLICSDEASGCHYGALTCGSCKVFFKRAAEGKQKYLCASRNDCTIDKLRRKNCPSCRLKKCFEVGMTLGARKLRKIGQMKGPDEVGAVQGPSETVQCLSPKPNLTFHSQLIFLNILEAIEPEVVNAGHDHGQPDSAAALLTSLNELGERQLVKVVKWAKGLPGFRNLHVDDQMTVIQHTWMGVMVFALGWRSYKNANARMLYFAPDLVFNDRRMHVSSMYEHCVQMKHLSQEFVLLQVTQEEFLCMKALLLFSVIPVEGLKSQKYFDELRLTYINELDRLINYGRKTNCAMRFQQLTRLMDSLQPVVQKLHQFTFDLFVQARSLPTKVSFPEMIAEIISVQVPKMLAGLSKPILFHK